(19) United States
(12) Patent Application Publication
(10) Pub. No.: US 2010/0136636 A1
(43) Pub. Date: Jun. 3, 2010

(54) PROCESS FOR SELECTIVE PRODUCTION OF THEAFLAVIN

(76) Inventor: Masumi Takemoto, Shizuoka (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/452,557

(22) PCT Filed: Jul. 11, 2008
(86) PCT No.: PCT/JP2008/062579

§ 371 Date: Jan. 28, 2010

(30) Foreign Application Priority Data
Jul. 11, 2007 (JP) 2007-182217

(51) Int. Cl. C12P 17/06; C12N 5/04

(52) U.S. Cl. 435/125; 435/410

(57) ABSTRACT

The present invention provides a method for selective production of theaflavin in large amounts at high yield, and in an easy and inexpensive manner. Specifically, it relates to a method for selective production of theaflavin whereby a processed plant extract containing epicatechin, epigallocatechin, epicatechin-3-O-gallate and epigallocatechin-3-O-gallate is combined with a plant cell culture having peroxidase activity for selective production of theaflavin.

### TECHNICAL FIELD

The present invention relates to a method for selective production of theaflavin (TF). More specifically, the invention relates to a method for selective production of theaflavin at high yield, by reaction of epicatechin, epigallocatechin, epicatechin-3-O-gallate and epigallocatechin-3-O-gallate in a plant extract in the presence of a plant cell culture having peroxidase activity.

### BACKGROUND ART

Theaflavin is a compound that has been known as a red pigment in black tea, and in recent years its has been shown to exhibit various physiological effects including antioxidant, glucose-lowering and antibacterial effects, for which reason it has become recognized as potentially useful not only as a natural coloring agent but also as a bioactive substance, and further research on the compound is expected.

Theaflavin has three different galloyl esters, namely theaflavin-3-O-gallate (TF3-G), theaflavin-3′-O-gallate (TF3′-G) and theaflavin 3,3′-di-O-gallate (TFDG). Their chemical structures are shown below as chemical formulas a-d.

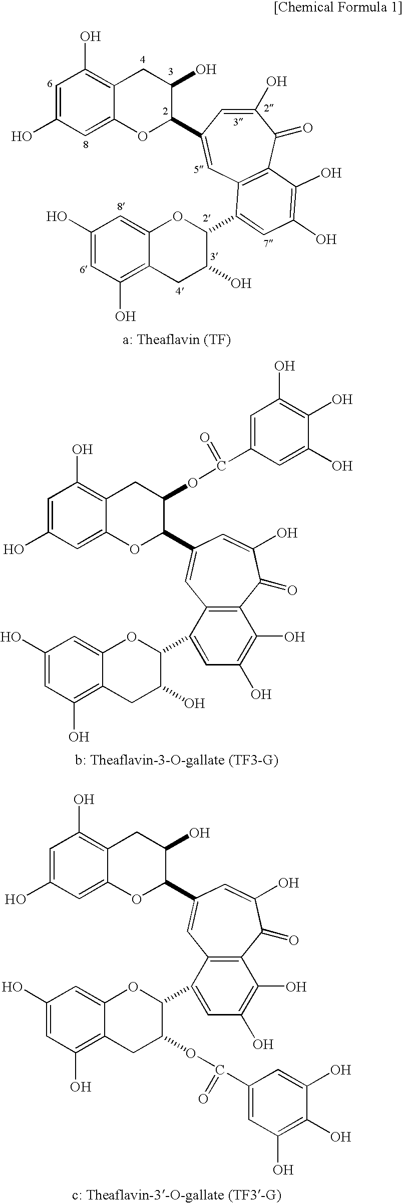

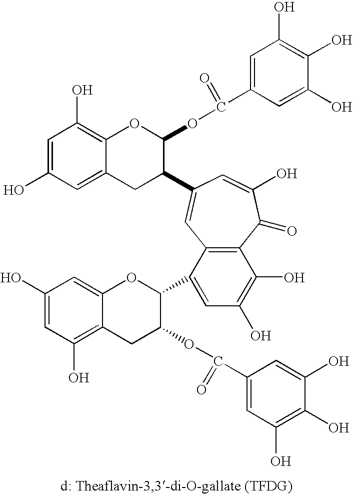

The approximate proportions of these four compounds in black tea are TF: 0.08 wt %, TF3-G: 0.3 wt %, TF3′-G: 0.2 wt % and TFDG: 0.4 wt %.

Theaflavin is known to be produced by enzymes in tea leaves during production of black tea, and is generally obtained by extraction from black tea. However, the content in black tea is extremely low, and for example, it is only present in a total of about 0.08 wt % in black tea. It has therefore been difficult to obtain a sufficiently usable amount simply by extraction from black tea leaves.

The theaflavin biosynthetic pathway involves biosynthesis from epicatechin (EC) and epigallocatechin (EGC), as represented by the chemical reaction formula (I) and (II) below (Non-patent document 1).

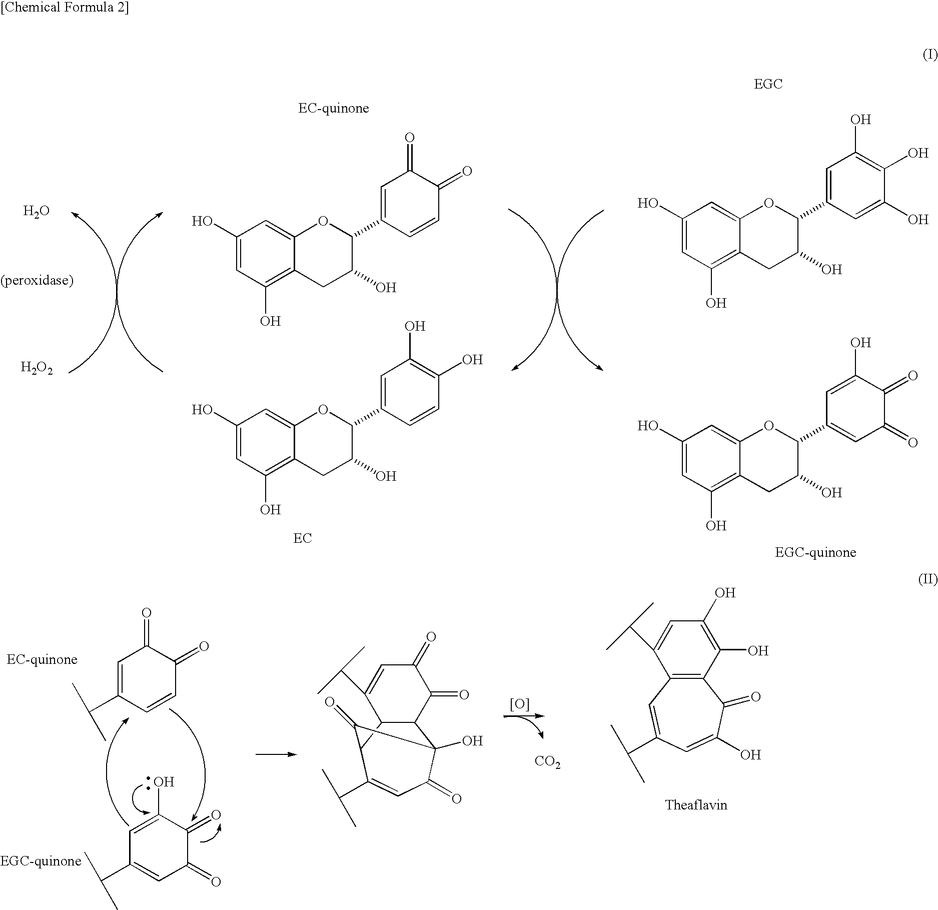

First, epicatechin (EC) is oxidized by polyphenol oxidase or peroxidase to EC-quinone, and the EC-quinone in turn oxidizes epigallocatechin (EGC) to produce EGC-quinone. Michael addition of the EGC-quinone obtained by these oxidation steps to EC-quinone and subsequent carbonylation produces a 3-membered ring intermediate, which is further oxidized and decarboxylated to form a troponoid skeleton, thus producing theaflavin.

Similarly, TF3-G is biosynthesized from epicatechin (EC) and epigallocatechin-3-O-gallate (EGCG) as shown below.

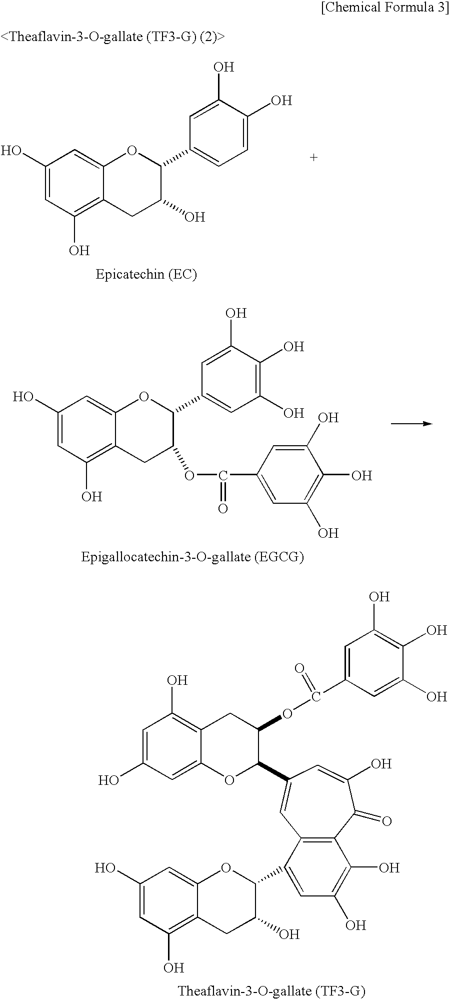

Similarly, TF3′-G is biosynthesized from epicatechin-3-O-gallate (ECG) and epigallocatechin (EGC) as shown below.

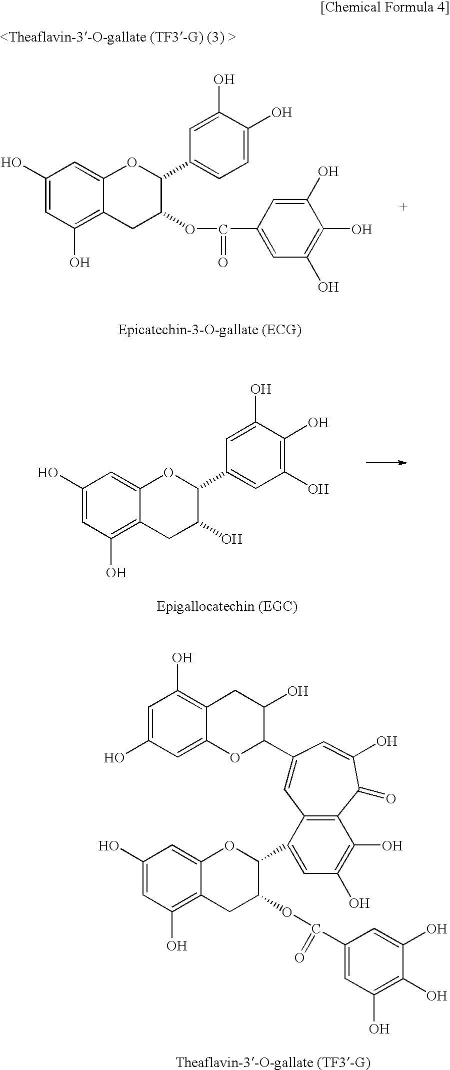

Likewise, TFDG is biosynthesized from epicatechin-3-O-gallate (ECG) and epigallocatechin-3-O-gallate (EGCG) as shown below.

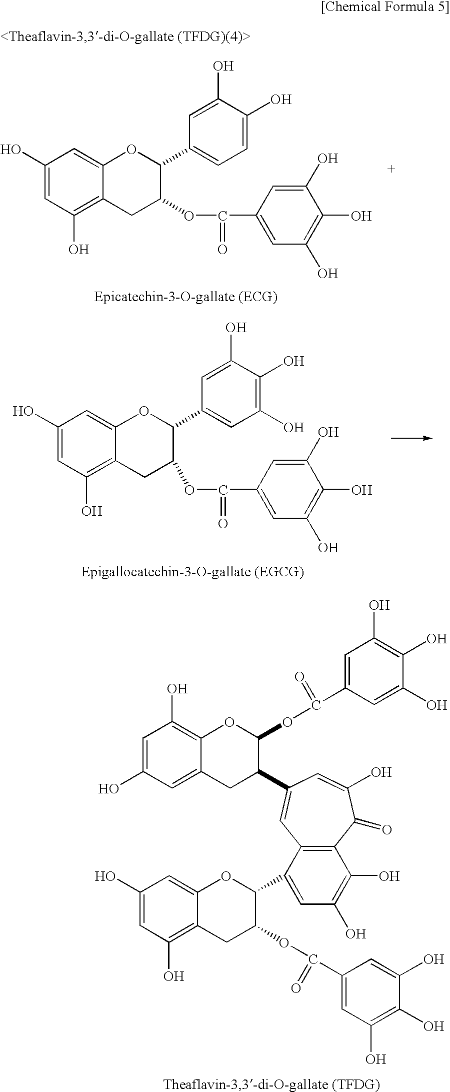

As regards production of theaflavin by oxidation reaction of epicatechin and epigallocatechin, Patent document 1 discloses a method for producing theaflavin and its three galloyl esters by combining plant extracts containing polyphenol oxidase (a water extract of the fruit, immature fruit, leaves, rhizome, root, fruit body, seeds and buds of different plants) with green tea extract. However, the method in Patent document 1 produces theaflavin and its three galloyl esters, instead of selective production of theaflavin alone as according to the present invention.

Patent document 2 discloses a method for producing a theaflavin-rich tea extract by treating a green tea (green leaf tea) slurry with tannase and then fermenting the slurry.

The method of Patent document 2 produces a theaflavin-rich tea extract from epicatechin (EC) and epigallocatechin (EGC), by first converting the epicatechin-3-O-gallate (ECG) and epigallocatechin-3-O-gallate (EGCG) to epicatechin (EC) and epigallocatechin (EGC), respectively, using tannase, in order to prevent production of the galloyl esters (TF3-G, TF3′-G, TFDG), and then blowing air or oxygen into the dhool (green tea leaves softened and rendered flaccid by immersion in a liquid) slurry for fermentation.

However, the method of Patent document 2 has required reaction with tannase under nitrogen, in order to prevent production of theaflavin galloyl esters by fermentation before conversion of the epicatechin-3-O-gallate (ECG) and epigallocatechin-3-O-gallate (EGCG) to epicatechin (EC) and epigallocatechin (EGC) by tannase. In addition, while TF biosynthesis occurs with 1 mol of EGC and 1 mol of EC, the differing oxidation rates introduce a restriction in which green tea with an EGC:EC molar ratio of 3:1 is preferred for use. Addition of tannase has also been a cost-increasing factor in the production of theaflavin.

The present inventors have previously reported that cultured tea cells have high peroxidase activity, and have studied their application for production of various drugs (Non-patent document 2). We have also disclosed a method for producing theaflavin using cultured plant cells with high peroxidase activity, such as cultured tea cells, and epicatechin (EC) and epigallocatechin (EGC) (Patent document 3). However, because of the high cost of epicatechin (EC) and epigallocatechin (EGC), their use has led to increased cost for theaflavin production.

It has therefore been desired to develop an inexpensive, large-scale, simple method for producing theaflavin that can be applied for industrial mass production.

[Patent document 1] Japanese Unexamined Patent Publication No. 2002-95415
[Patent document 2] Japanese Unexamined Patent Publication HEI No. 11-225672
[Patent document 3] Japanese Unexamined Patent Publication No. 2007-143461
[Non-patent document 1] Takahashi Tanaka, Chie Mine, Kyoko Inoue, Miyuki Matsuda and Isao Kouno, J. Agric. Food Chem. 2002, 50. 2142-2148.
[Non-patent document 2] Masumi Takemoto, Youichi Aoshima, Nikolay Stoynov and James Peter Kutney, Tetrahedron Letters, 43, 6915-6917, 2002

### DISCLOSURE OF THE INVENTION

### Problems to be Solved by the Invention

It is an object of the present invention to provide a method for selective production of theaflavin in large amounts at high yield, and in a simple and inexpensive manner.

### Means for Solving the Problems

As a result of much diligent research in light of the above, the present inventors have found that theaflavin is selectively produced when a processed plant extract containing epicatechin, epigallocatechin, epicatechin-3-O-gallate and epigallocatechin-3-O-gallate is combined, with a plant cell culture having peroxidase activity. It was further found that the selective production of theaflavin is achieved at high yield simply by replenishing epicatechin in the plant extract.

That is, the present invention provides a method for producing theaflavin according to the following (1)-(7).

(1) A method for selective production of theaflavin whereby a processed plant extract containing epicatechin, epigallocatechin, epicatechin-3-O-gallate and epigallocatechin-3-O-gallate is combined with a plant cell culture having peroxidase activity for selective production of theaflavin.

(2) The method for producing theaflavin according to (1) above, wherein the plant is tea leaves and/or stems.

(3) The method for producing theaflavin according to (1) above, wherein the processed plant extract is an unfermented tea extract.

(4) The method for producing theaflavin according to any one of (1) to (3) above, wherein the plant cell culture is a culture of tea cells.

(5) The method for producing theaflavin according to any one of (1) to (4) above, characterized by further adding hydrogen peroxide.

(6) The method for producing theaflavin according to any one of (1) to (5) above, characterized by further adding epicatechin.

(7) Cultured plant cells for the method for producing theaflavin according to any one of (1) to (6) above.

### Effect of the Invention

The present invention allows selective production of TF alone in a short period of time using epicatechin, epigallocatechin, epicatechin-3-O-gallate and epigallocatechin-3-O-gallate as starting materials in the presence of oxygen, using a plant cell culture without addition of tannase or treatment under nitrogen, even when epicatechin-3-O-gallate and epigallocatechin-3-O-gallate are present in addition to epicatechin and epigallocatechin. It is thereby possible to use the inexpensive material of bancha (coarse green tea) as the material for theaflavin production, thus allowing selective production of theaflavin without the need for addition of expensive tannase or complex treatment under nitrogen, so that theaflavin can be produced in an inexpensive manner.

In addition, since the amount of theaflavin production can be increased simply by replenishment of epicatechin, it is possible to efficiently and inexpensively produce large amounts of theaflavin.

### BRIEF DESCRIPTION OF THE DRAWINGS

FIG. 1 shows HPLC analysis for the reaction mixture of Example 1 at the start of the reaction (0 min). The HPLC analysis conditions were as described in Example 1. The analysis sample was a portion of the reaction mixture at the start of the reaction (0 min), directly supplied for HPLC analysis.

Production of gallic acid by hydrolysis of ECG and EGCG was observed during 24 hours of aqueous extraction of bancha.

FIG. 2 shows HPLC analysis of the reaction mixture of Example 1 at 356 minutes after the start of reaction. The HPLC analysis conditions were as described in Example 1. The analysis sample was a portion of the reaction mixture at 356 minutes after start of the reaction, directly supplied for HPLC analysis.

FIG. 3 shows HPLC analysis of the reaction mixture of Example 2 at 9 minutes after the start of reaction. The HPLC analysis conditions were as described in Example 1. The analysis sample was a portion of the reaction mixture at 9 minutes after start of the reaction, directly supplied for HPLC analysis. Peaks for EC, ECG, EGC and EGCG are virtually absent, but a peak for TF is observed.

FIG. 4 shows HPLC analysis of the reaction mixture of Example 2 at 28 minutes after the start of reaction. The HPLC analysis conditions were as described in Example 1. The analysis sample used was an extract obtained by ethyl acetate extraction of the reaction mixture at 28 minutes after start of the reaction. A peak is observed for theanaphthoquinone, and the peak for theaflavin is reduced.

FIG. 5 shows HPLC analysis of the reaction mixture of Example 5 at 5 minutes after the start of reaction. The analysis sample was a portion of the reaction mixture at 5 minutes after start of the reaction, directly supplied for HPLC analysis.

FIG. 6 shows HPLC analysis of the ethyl acetate extract of the reaction mixture upon completion of the reaction (12 min) in Example 5. The analysis sample used was an extract mixture obtained by ethyl acetate extraction of the reaction mixture upon completion of the reaction (12 min).

### BEST MODE FOR CARRYING OUT THE INVENTION

Embodiments of the invention will now be described.

The epicatechin, epigallocatechin, epicatechin-3-O-gallate and epigallocatechin-3-O-gallate used for the invention are the following compounds.

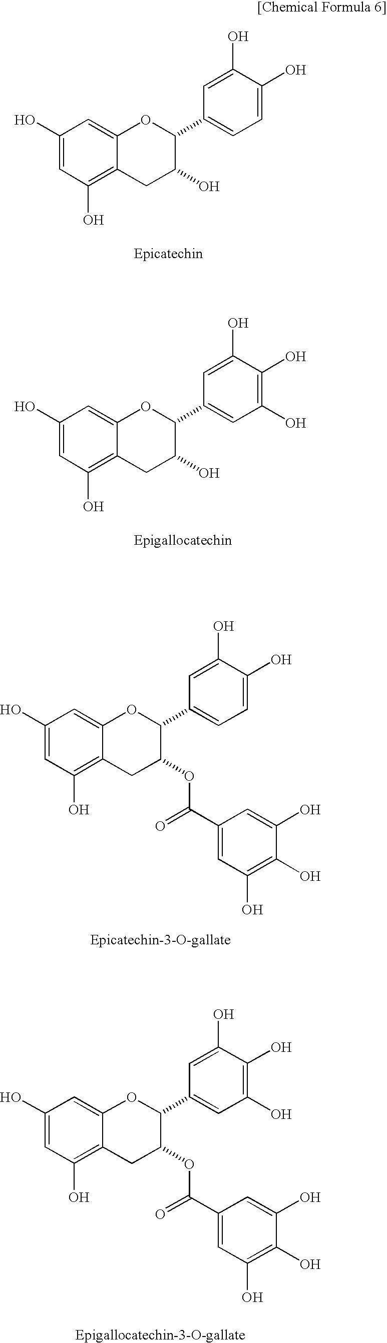

The type of plant used for the invention is not restricted so long as it contains epicatechin, epigallocatechin, epicatechin-3-O-gallate and epigallocatechin-3-O-gallate, and the plant may be an entire plant or a section thereof (seedling, leaf, cotyledon, stem, fruit or the like). The preferred type of plant is tea (Camellia sinensis, Camellia assamica). The preferred plant parts are seedlings, leaves, cotyledons, stems and fruits, more preferably leaves, cotyledons and stems, and most preferably leaves and stems.

A processed plant means a plant that has been treated by heating or the like to inactivate the oxidizing enzymes in the plant, and it is not particularly restricted, although preferably it is heat-treated tea and more preferably processed tea such as unfermented tea used for beverages.

The method and conditions of the treatment are not particularly restricted so long as the treatment allows inactivation of oxidizing enzymes in the plant, but steaming and/or roasting are preferred.

An “oxidizing enzyme” is an enzyme that synthesizes TF from EC and EGC, TF3-G from EC and EGCG, TF3′-G from ECG and EGC or TFDG from ECG and EGCG, and although it is not restricted it is preferably polyphenol oxidase and/polyphenol oxidase or peroxidase.

Unfermented tea refers to tea leaves or stems that have been heat treated by steaming and/or roasting of the tea leaves or stems for inactivation of the oxidizing enzymes in the tea, and green tea may be included as an example. The green tea is not limited, and for example, it includes Japanese green tea provided as a beverage in Japan and prepared by heat treatment of tea leaves or stems by steaming and the like to inactivate the oxidizing enzymes in the tea, and Chinese green tea provided as a beverage in China and prepared by heat treatment of tea leaves or stems by roasting and the like to inactivate the oxidizing enzymes in the tea. Japanese green tea also includes sencha, kabusecha, gyokuro, powdered tea, yonkoncha, bancha and hojicha, based on differences in the tea materials (cultivation method, leaves and stems) and in the methods of preparation. For epicatechin, epigallocatechin, epicatechin-3-O-gallate and epigallocatechin-3-O-gallate, it is known that catechin, gallocatechin, catechin-3-O-gallate and gallocatechin-3-O-gallate (called these as isomerized forms) are present. These isomerized forms are preferably absent for selective production of theaflavin. The preferred green tea used for the invention are Chinese green tea and Japanese green tea. More preferred are Japanese green tea such as sencha and bancha. Bancha is particularly preferred because it has a low isomerization content and is also inexpensive.

Sencha is processed tea exclusively from tea leaves picked from late April to early May in Japan (the initial sprouts or young leaves of the year, called “ichibanchaba”) and tea leaves grown after picking (the sprouts or young leaves, called “nibanchaba”) as materials. Bancha refers to processed tea from tea leaves or stems other than ichibanchaba or nibanchaba, but ichibanchaba or nibanchaba may also be included in the material. Thus, the tea leaves and/or stems used for the invention are preferably the tea leaves and/or stems used as starting materials for sencha or bancha.

For example, green tea known as “sencha” in Japan is prepared by steaming only picked ichibanchaba or nibanchaba to inactivate the oxidizing enzymes in the tea leaves, and then cooling the tea leaves, kneading the tea leaves, and drying the tea leaves. Green tea known as “bancha” in Japan is produced by the same procedure as sencha, but since tea leaves or stems other than ichibanchaba and nibanchaba are used as material, the steaming for inactivation of the oxidizing enzymes is stronger than for sencha (the steaming time is longer). The steaming time for inactivation of the oxidizing enzymes is not particularly restricted and will depend on the material (that is, the sprouts, young leaves or grown leaves or stems) and amount of tea used.

The invention includes green tea that is equivalent to sencha or bancha. The invention also includes green tea that is equivalent to Chinese green tea.

The plant extract used for the invention is obtained by extraction from a plant using an aqueous solvent, organic solvent or a mixed solvent thereof. Thus, a processed plant extract is one obtained by extraction from a processed plant using an aqueous solvent, organic solvent or a mixed solvent thereof.

An aqueous solvent used for extraction is not particularly restricted so long as it is a solvent containing water and is capable of extracting epicatechin, epigallocatechin, epicatechin-3-O-gallate and epigallocatechin-3-O-gallate from a plant, but it is preferably water, phosphate buffer, citrate buffer or the like. Water is more preferred.

An organic solvent used for extraction is not particularly restricted so long as it is an organic solvent capable of extracting epicatechin, epigallocatechin, epicatechin-3-O-gallate and epigallocatechin-3-O-gallate, but it is preferably ethyl acetate; an alcohol such as methanol or ethanol; acetone; or an ether such as diethyl ether. It is preferably ethyl acetate; an alcohol such as methanol or ethanol; or acetone.

The extraction method may be any method whereby epicatechin, epigallocatechin, epicatechin-3-O-gallate and epigallocatechin-3-O-gallate elute into the solvent, and there are no restrictions on the conditions such as the temperature and time.

The plant cell culture used for the invention is obtained by transferring a heterogeneous undifferentiated proliferative cell mass (callus) artificially induced from the adult plant body into liquid culture, and forming a culture suspension (cells and/or culture solution) comprising cultured cells that proliferate stably and rapidly.

Since the plant cell culture used for the invention uses successively cultured plant cells, it is advantageous in that it can be provided at any time for reaction and is highly mobile, as well as inexpensive.

Plant cells generally contain peroxidases, and any plant cells having peroxidase activity may be used as the plant for the plant cell culture according to the invention, but preferred plants for the plant cell culture used for the invention include tobacco (N. tabacum), carrot (D. carota), nichinichiso (C. roseus), tea (Camellia sinensis, Camellia assamica) and podophyllum (P. peltatum). There may also be used Japanese radish, cucumber, eggplant, horseradish, soybean and the like.

Cells derived from tea (Camellia sinensis, Camellia assamica) are particularly preferred among these because of their high peroxidase activity and suitability for the method of the invention. The tea variety may be Yabukita, Okuhikari, Yama-no-ibuki, Sayamakaori, Kanayamidori, Surugawase or the like, and the peroxidase activity will differ depending on the variety. These varieties are cultivated for beverage use in Japan and are commercially available. Most preferred are Yabukita cotyledons, Sayamakaori cotyledons and Surugawase stems.

The plant cells for the plant cell culture may be from the tissue of any part (seedling, leaf, cotyledon, stem, root, etc.). Seedling, leaf, cotyledon and stem are preferred, and seedling and cotyledon are more preferred.

The plant cell culture used for the invention is prepared by using a segment of live tissue of the plant cells to induce a callus based on a common callus-forming method known in the prior art, transferring and culturing it in culture medium. The cell culture of the invention may be a cell culture obtained by culturing the callus in solid culture medium, or a culture suspension of cultured cells obtained using liquid medium.

Culturing of the segment is accomplished by first sterilizing the segment and culturing it on solid culture medium such as agar medium to induce a callus. The induced callus is fully grown on the same solid culture medium or in liquid medium. Sterilization of the segment is accomplished by ethanol surface sterilization, hypochlorous acid salt treatment, sterilized distillation ion-exchanged water rinsing, or the like.

The culture medium may be basal medium containing inorganic salts or vitamins, such as Murashige-Skoog (MS) medium or Gamborg's B5 (B5) medium, or Nitch+Nitch, to which are added saccharides such as sucrose, maltose or glucose as carbon sources, ammonium nitrate, potassium nitrate, ammonium sulfate or ammonium tartrate as nitrogen sources, as well as casamino acids, amino acids, peptone, yeast cells, yeast extract, malt extract and the like.

There may also be added vitamins such as nicotinic acid, nicotinamide, thiamine, folic acid and biotin, or inositol, adenylic acid, guanylic acid, cytidylic acid, thymidylic acid, cyclic AMP and the like, or minerals such as iron, manganese, zinc, boron, iodine, potassium, cobalt, magnesium, molybdenum, phosphorus and copper.

The basal medium may also employ appropriately plant hormones such as 2,4-dichlorophenoxyacetic acid (2,4D), naphthaleneacetic acid, indolebutyric acid, indoleacetic acid, benzyladenine and kinetin. A particularly preferred plant hormone is 2,4-dichlorophenoxyacetic acid (2,4D).

If necessary, it may have a composition including agar or the like.

The culturing conditions may be appropriately determined according to the medium and the sorts of additives used, but preferred conditions are under darkness, preferably at about 15-40° C. and more preferably about 25-27° C., for approximately 10-14 days. In this case, a solid culture medium added agar may be used for stationary culturing, but in order to achieve homogeneous cultured cells it is preferred to use liquid medium for culturing while agitating at a rotational speed of about 50-170 rpm and more preferably about 100-110 rpm, to obtain a culture suspension of the cultured cells.

The amount of additives used such as plant hormones and carbon sources may also be appropriately determined according to the culture medium and culturing conditions used, but the concentration of 2,4-dichlorophenoxyacetic acid (2,4D) used as a plant hormone is preferably 0.05-20 mg/L and more preferably 0.2-10 mg/L. If the 2,4D concentration is too low, the peroxidase activity will tend to be reduced.

The sucrose concentration, when using sucrose as a carbon source, is preferably 10,000-130,000 mg/L and more preferably 30,000-70,000 mg/L. If the sucrose concentration is too low, the peroxidase activity will tend to be reduced.

The plant cell culture obtained in this manner contains the cultured cells of the plant and culture solution. Since peroxidase often effuses from cultured plant cells into culture solutions, the culture solution will also have peroxidase activity similar to the cultured plant cells. According to the invention, the culture solution alone, obtained by removing the cultured plant cells from the plant cell culture, may also be effectively used for production of theaflavin. The industrial utility of the production method of the invention may therefore widen greatly.

According to the invention, the cultured cells may be immobilized for use. This provides an additional advantage in that the culture of the immobilized cultured cells may be added to a reaction system after adjustment to the appropriate amount like an ordinary reagent, and recovered by an easy procedure for reuse. When a culture of immobilized cultured cells is used the reaction will generally tend to slow, but with each reuse the enzyme activity will increase, thus also increasing the conversion ratio, and finally the yield and selectivity will increase to obtain highly pure theaflavin.

The immobilizing support is not restricted, and agar, agarose, w-carrageenan, alginic acid, polyacrylamide, polyurethane, photo-crosslinkable resins, photosensitive resins and the like may be used. The shape of the immobilizing support may be beads, blocks, cylinders, a film, or the like. The immobilizing method may be any method known in the prior art.

In order to increase the peroxidase activity of the immobilized cultured cells, it is preferred to agitate the immobilized cultured cells in culture medium after preparation of them for a certain period, preferably 5 days or longer. More preferably, they are agitated for 5 days or longer in culture medium with an increased sucrose concentration, specifically in culture medium containing sucrose at 0.4 M or greater concentration. It is also effective to prepare at increased concentration of the immobilizing agent (calcium alginate), specifically 1.1 wt % or greater. In addition, it is effective to allow it to stand for a certain period (approximately 30 minutes) in a 20 vol % dimethyl sulfoxide aqueous solution after preparing at the immobilizing agent (calcium alginate or strontium alginate) concentration of 0.6 wt % or greater. It is also effective to allow it to stand for a certain period (approximately 30 minutes) in a 10 vol % dimethyl sulfoxide aqueous solution after preparing at the immobilizing agent concentration of 0.6 wt % or greater, and then react it in a hexane solvent.

The peroxidase activity used for the invention is defined as activity of converting pyrogallol to purpurogallin, as shown below.

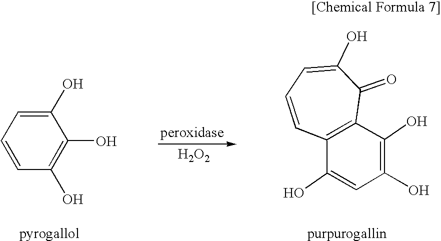

There are no particular restrictions on the strength of peroxidase activity of the plant cell culture used for the invention, and it is preferably 0.5 unit/mL, and more preferably 6-20 unit/mL.

The mixing of a processed plant extract with the plant cell culture according to the invention means combining the epicatechin, epigallocatechin, epicatechin-3-O-gallate and epigallocatechin-3-O-gallate in the plant extract with the plant cell culture, to selectively produce theaflavin.

The mixing of the processed plant extract with plant cell culture used for the invention is preferably carried out in an aqueous solvent. When the plant extract has been extracted with an aqueous solvent, the plant extract and plant cell culture (cells and/or culture solution) may be directly mixed. When the plant extract has been extracted with an organic solvent, on the other hand, it may be mixed with the plant cell culture (cells and/or culture solution) after removing the organic solvent and dissolving in an aqueous solvent. When the plant cell culture contains culture solution, the plant extract from which the organic solvent has been removed may be added to and mixed with the plant cell culture solution.

The mixture is preferably agitated after and/or during mixing. The preferred agitation rate is about 50-170 rpm and more preferably about 100-110 rpm, although this is not restrictive.

The aqueous solvent in the mixture of the processed plant extract and plant cell culture is not restricted so long as it is an aqueous solvent in which peroxidases are active, and there may be included water, plant cell-culturing medium, plant cell-cultured solution, phosphate buffer, citrate buffer and the like. Water, plant cell-culturing medium and plant cell-cultured solution are preferred, and water is more preferred. Water will eliminate the need for disposal of phosphoric acid or the like in the waste solution with industrialization, thus facilitating disposal of the waste solution. The buffer concentration is about 0.3 M-0.001 M, preferably 0.2 M-0.01 M, and more preferably about 0.1 M-1/15 M.

The mixing proportion of the processed plant extract and plant cell culture used for the invention is not particularly restricted, but it is preferably 5-250 units, more preferably 10-100 units and most preferably 30-40 units of peroxidase in the plant cell culture, with respect to a total amount of 6 mg EC and ECG or a total amount of 6 mg EGC and EGCG in the plant extract.

The mixing for the invention may be carried out at a temperature of preferably 15-70° C. and more preferably 25-40° C., for a period of preferably 5-540 minutes, more preferably 6-30 minutes and most preferably 7-15 minutes, but the mixing temperature and mixing time may be appropriately selected in consideration of the other conditions including the amounts of material and enzyme. The theaflavin synthesis reaction from epicatechin (EC) and epigallocatechin (EGC) in the mixture used according to the invention is more rapid than theaflavin-3-O-gallate (TF3-G) synthesis reaction from EC and EGCG, theaflavin-3′-O-gallate (TF3′-G) synthesis reaction from ECG and EGC or theaflavin-3,3′-di-O-gallate (TFDG) synthesis reaction from ECG and EGCG, and the shortened reaction time therefore allows more selective production of theaflavin. This is because the cultured tea cells used for the invention have peroxidase activity that can produce theaflavin more rapidly than production of theaflavin gallo esters, in the presence of hydrogen peroxide. Moreover, the shortened reaction time can also help prevent decomposition of the produced theaflavin.

According to the invention, selective production of theaflavin is specific synthesis of theaflavin (TF) from EC and EGC in the presence of epicatechin (EC), epigallocatechin (EGC), epicatechin-3-O-gallate (ECG) and epigallocatechin-3-O-gallate (EGCG) as materials, with virtually no synthesis of theaflavin-3-O-gallate (TF3-G) from EC and EGCG, theaflavin-3′-O-gallate (TF3′-G) from ECG and EGC or theaflavin-3,3′-di-O-gallate (TFDG) from ECG and EGCG. Specifically, this means that the proportion of TF in the product (total of TF, TF3-G, TF3′-G and TFDG) upon completion of the reaction is 90 wt % or greater, preferably 95 wt % or greater and even more preferably 99 wt % or greater.

According to the invention, hydrogen peroxide may be added if necessary to the mixture of the processed plant extract and the plant cell culture (cells and/or culture solution).

Since synthesis of theaflavin from epicatechin (EC) and epigallocatechin (EGC) is oxidation reaction by peroxidases, hydrogen peroxide (H₂O₂) is preferably copresent in the reaction system. However, hydrogen peroxide is not essential in some cases. Since some plant cells produce hydrogen peroxide themselves, theaflavin synthesis is possible without addition of hydrogen peroxide if such plant cells are used as the cultured cells of the invention.

When hydrogen peroxide is included together with the plant cell culture, hydrogen peroxide water adjusted to an appropriate concentration is added to the mixing system together with the plant cell culture. The concentration of the hydrogen peroxide water used for addition is preferably 0.1-30 vol % and more preferably 0.3-10 vol %.

The preferred total amount of hydrogen peroxide in the mixture is 1-30 mg and more preferably 3-10 mg with respect to 0.069 mmol as the total of EC and ECG in the mixture.

The tea leaves used for the invention will generally contain 0.9 wt % epicatechin, 0.8 wt % epicatechin-3-O-gallate, 3.5 wt % epigallocatechin and 3.8 wt % epigallocatechin-3-O-gallate. Since equimolar amounts of epicatechin and epigallocatechin react during synthesis of theaflavin, a lack of epicatechin with respect to epigallocatechin will result.

According to the invention, the epicatechin and epigallocatechin to serve as materials for theaflavin production are not only epicatechin and epigallocatechin originally present in the plant extract, since epicatechin-3-O-gallate and epigallocatechin-3-O-gallate are also hydrolyzed at their gallate groups to epicatechin and epigallocatechin as illustrated by scheme 1, and thus serve as materials for theaflavin production.

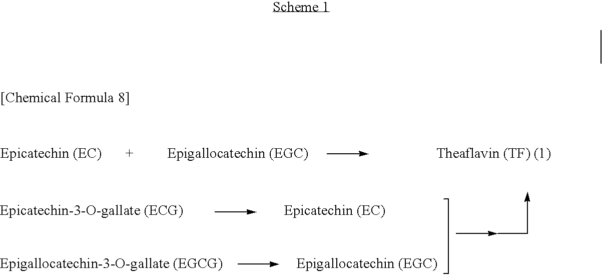

The total amount of epicatechin and epicatechin-3-O-gallate (1.7 wt %) is much lower than the total amount of epigallocatechin and epigallocatechin-3-O-gallate (7.3 wt %).

Consequently, epicatechin is added according to the invention for external replenishment of epicatechin that is deficient with respect to epigallocatechin (or epigallocatechin-3-O-gallate) in the plant extract, allowing the unreacted epigallocatechin (or epigallocatechin-3-O-gallate) to participate in theaflavin synthesis and thereby increase production of theaflavin from the plant extract. The amount of replenished epicatechin is an amount such that the total of epicatechin and epicatechin-3-O-gallate in the mixture of the plant extract and plant cell culture is at least an equimolar amount with respect to the total of epigallocatechin and epigallocatechin-3-O-gallate, and preferably such that the total (molar) amount of epicatechin and epicatechin-3-O-gallate:the total (molar) amount of epigallocatechin and epigallocatechin-3-O-gallate=1.0-2.0:1.0 and more preferably 1.1-1.5:1.0.

After the synthesis reaction has completed, the theaflavin is separated from the reaction mixture and purified. The separation may be accomplished by an extraction procedure. Chloroform is used for initial extraction to remove the caffeine and lipids, and then a common extraction solvent, i.e. an ester such as ethyl acetate or an ether such as diethyl ether, may be used for extraction of theaflavin.

After the extraction procedure, the theaflavin dissolved in the extraction solvent is separated by distilling off the solvent by concentration under reduced pressure, for example, after dewatering with magnesium sulfate or the like. This produces an extraction residue containing theaflavin.

The theaflavin-containing extraction residue obtained after the extraction procedure above may be separated and purified by known techniques such as molecular sieving column chromatography or column chromatography using an ion exchange resin or the like. Such a method can yield purified theaflavin. Production of theaflavin can be analyzed using high performance liquid chromatography (HPLC), thin-layer chromatography, gas chromatography or mass spectrometry or the like.

Since essentially theaflavin alone is produced according to the method of the invention, it is possible to obtain the target substance at high purity by solvent extraction of the reaction mixture.

The present invention will now be explained in greater detail with reference to examples, with the understanding that the invention is not meant to be limited to these examples.

### Example 1

### Preparation of Plant Cell Culture

For this experiment, adult tea cells (tea variety: Sayamakaori, tea section: seedling) were used as plant cells for preparation of a tea cell culture in the following manner. Specifically, a callus of cultured tea cells grown on agar medium was transferred to Gamborg's B5 (B5) medium (100 mL per flask) containing 2,4-dichlorophenoxyacetic acid and sucrose. This was then cultured under dark conditions at 25° C. while shaking at 110 rpm for approximately 10 days until homogeneity of the cultured cells, to prepare a tea cell culture (Camellia sinensis cell culture). The peroxidase activity of the tea cell culture was 15.5 unit/mL.

Also, adult cells of tobacco (N. tabacum) and carrot (D. carota) were used for culturing in media having the composition shown in Table 1 instead of tea, and plant cell cultures including these cultured cells were prepared in the same manner as Example 1.

The peroxidase activities of these cell cultures were tobacco (N. tabacum)=9.6 unit/mL, carrot (D. carota)=8.0 unit/mL.

The composition of the B5 medium is shown in Table 1.

TABLE 1

| | C. sinensis | | N. tabacum | | D. carota | |
|---|---|---|---|---|---|---|
| NH₄NO₃ | — | | 1650 | mg | 1650 | mg |
| KNO₃ | 2830 | mg | 1900 | mg | 1900 | mg |
| CaCl₂•2H₂O | 150 | mg | 440 | mg | 440 | mg |
| MgSO₄•7H₂O | 250 | mg | 370 | mg | 370 | mg |
| KH₂PO₄ | 280 | mg | 170 | mg | 170 | mg |
| H₃BO₃ | 3 | mg | 6.2 | mg | 6.2 | mg |
| MnSO₄•4H₂O | 10 | mg | 22.3 | mg | 22.3 | mg |
| ZnSO₄•7H₂O | 2 | mg | 8.6 | mg | 8.6 | mg |
| KI | 0.75 | mg | 0.83 | mg | 0.83 | mg |
| Na₂MoO₄•2H₂O | 0.25 | mg | 0.25 | mg | 0.25 | mg |
| CuSO₄•5H₂O | 0.025 | mg | 0.025 | mg | 0.025 | mg |
| CoCl₂•6H₂O | 0.025 | mg | 0.025 | mg | 0.025 | mg |
| Na₂-EDTA | 37.3 | mg | 37.3 | mg | 37.3 | mg |
| FeSO₄•7H₂O | 27.8 | mg | 27.8 | mg | 27.8 | mg |
| myoinositol | 100 | mg | 100 | mg | 100 | mg |
| glycine | 2 | mg | 2 | mg | 2 | mg |
| nicotinic acid | 0.5 | mg | 0.5 | mg | 0.5 | mg |
| thiamine•HCl | 1 | mg | 0.1 | mg | 0.1 | mg |
| pyridoxine•4HCl | 0.5 | mg | 0.5 | mg | 0.5 | mg |
| 2,4-dichlorophenoxy acetic | 1.25 | mg | 0.2 | mg | 1 | mg |
| acid | | | | | | |
| benzyladenine | 0.5 | mg | — | | — | |
| NaH₂PO₄•H₂O | 150 | mg | — | | 150 | mg |
| (NH₄)₂SO₄ | 464 | mg | — | | 134 | mg |
| sucrose | 50000 | mg | 30000 | mg | 30000 | mg |

### Measurement of Peroxidase Activity

A 1 ml enzyme-containing sample was added to 2 ml of 5% (WN) pyrogallol (Wako Pure Chemical Industries, Ltd.), 14 ml of H₂O, 1.0 ml of 0.5% H₂O₂ and 2.0 ml of 0.1 M pH 6.0 phosphate buffer under an atmosphere at approximately 25° C., and after vigorously agitating for 20 seconds, 1.0 ml of 2 M H₂SO₄ was added to quench the reaction. The reaction mixture was extracted twice with 25 ml of diethyl ether and adjusted to 100 ml with diethyl ether. The absorbance at 420 nm was measured. The amount of purpurogallin production was determined from a previously prepared purpurogallin calibration curve. The amount of enzyme producing 1 mg of purpurogallin under these conditions is defined as 1 unit of peroxidase activity, and the peroxidase activity (units) in 1 ml of sample was calculated from the amount of purpurogallin produced.

### Analysis of EC, ECG, EGC, EGCG and TF

EC, ECG, EGC, EGCG and TF were analyzed using an HPLC apparatus PU-980, UV-970 by JASCO, Co.) and an ODS120A (TOSHO, 4.6 mm×250 mm) column. The analysis samples were bancha water extract itself; ethyl acetate extract of bancha water extract; bancha ethyl acetate extract; reaction mixture itself; ethyl acetate extract of reaction mixture; or methanol elution fraction. The HPLC conditions were as follows: Solvent: acetonitrile:ethyl acetate: 0.05% H₃PO₄=21:3:76, flow rate: 1.0 ml/min, temperature: 25° C. Detection was performed at UV280 nm. Calibration curves were drawn using standard EC (Wako), ECG (Wako), EGC (Wako), EGCG (Wako) and TF (Nagara Science Co., Ltd.), and the amounts of EC, ECG, EGC, EGCG and TF in the samples were determined by calculation from the calibration curves.

Production of Theaflavin (TF) from Bancha Extract

After soaking 75 g of dry bancha (bancha by Ochano Mizuien) in 1500 ml of water for 24 hours for extraction, the tea leaf-filtered filtrate was obtained as 1840 ml of bancha extract (the amount of extract was 1840 ml as a result of tea leaf washing during filtration). The bancha extract contained 488.5 mg of EC, 122.4 mg of ECG, 556.5 mg of EGC and 610.5 mg of EGCG. After adding 45 ml of tea cell culture prepared as described above and 9 ml of 3% hydrogen peroxide water to the bancha extract, reaction was commenced while shaking the mixture at 110 rpm. At 4 hours after start of the reaction, 25 ml of tea cell culture and 4.5 ml of 3% hydrogen peroxide water were further added, at 5 hours after start of the reaction, 5 ml of tea cell culture and 1 ml of 3% hydrogen peroxide water were further added, and the reaction was quenched at 9 hours after start of the reaction. Extraction was performed 3 times with ethyl acetate after the reaction. The obtained ethyl acetate extract was concentrated with a rotary evaporator and then added to a Sephadex LH-20 column chromatograph (GE healthcare Bio-Sciences AB) previously equilibrated with methanol, and the red fraction was recovered by elution with methanol. Since theaflavin is red, recovery of the theaflavin fraction can be visually determined. As a result, 193 mg of theaflavin was obtained.

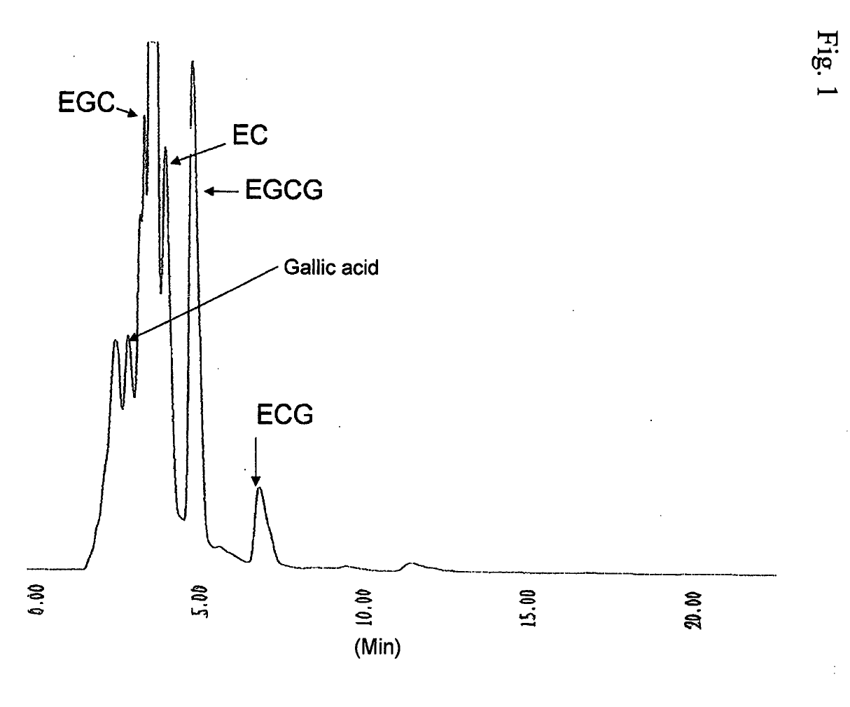

FIG. 1 shows the results of HPLC analysis of the reaction mixture at the start of the reaction (0 min). Peaks for EC, ECG, EGC and EGCG are observed, but no peak for theaflavin (TF) is observed.

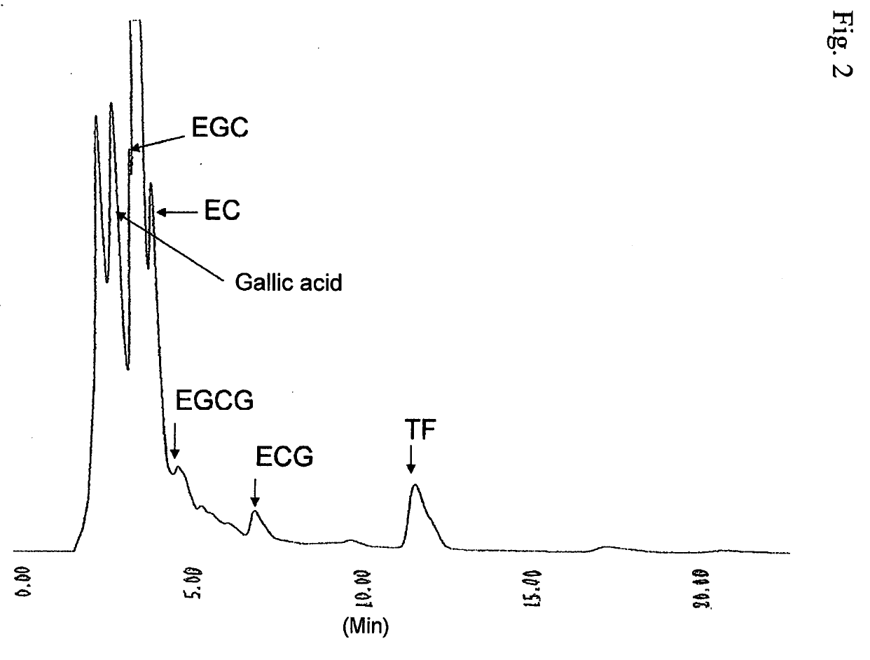

FIG. 2 shows the results of HPLC analysis of the reaction mixture at 356 minutes after start of the reaction. A peak for theaflavin (TF) is observed, but no galloyl ester peak is observed. This demonstrated selective production of theaflavin (TF). Additionally, a reduction in peaks for EGCG and ECG, especially a large reduction in the peak for EGCG was observed in the reaction mixture. An increase in the peak for gallic acid was also observed. Gallic acid is produced when EGCG is hydrolyzed and converted to EGC or when ECG is hydrolyzed and converted to EC, and therefore the increase in gallic acid suggests conversion from EGCG to EGC and from ECG to EC during the reaction. Thus, the lack of a large change in the peaks for EC and EGC despite consumption of EC and EGC by production of theaflavin (TF) permits to speculate that the consumed EC and EGC are supplied from ECG and EGCG, respectively.

### Example 2

For this experiment, the reaction starting material was changed from the bancha water extract to an ethyl acetate extract and the amount of the reaction starting material was reduced to examine production of theaflavin.

A 700 ml of bancha extract (a filtrate obtained by 24 hr extraction of 5 g of dry bancha with 600 ml of water and filtration removal of the tea leaves, with the tea leaf washing procedure during filtration resulting in 700 ml of extract) was extracted 3 times with ethyl acetate.

The ethyl acetate extract was concentrated to obtain 1.82 g of bancha extract. A 24.1 mg of the bancha extract (the 24.1 mg of bancha extract having an EC content of 4.3 mg, an ECG content of 1.1 mg, an EGC content of 4.9 mg and an EGCG content of 5.4 mg) was dissolved in 18.5 ml of water, and then 0.3 ml of 3% hydrogen peroxide and 1.2 ml of cultured tea cells were added and reaction was carried out for 28 minutes.

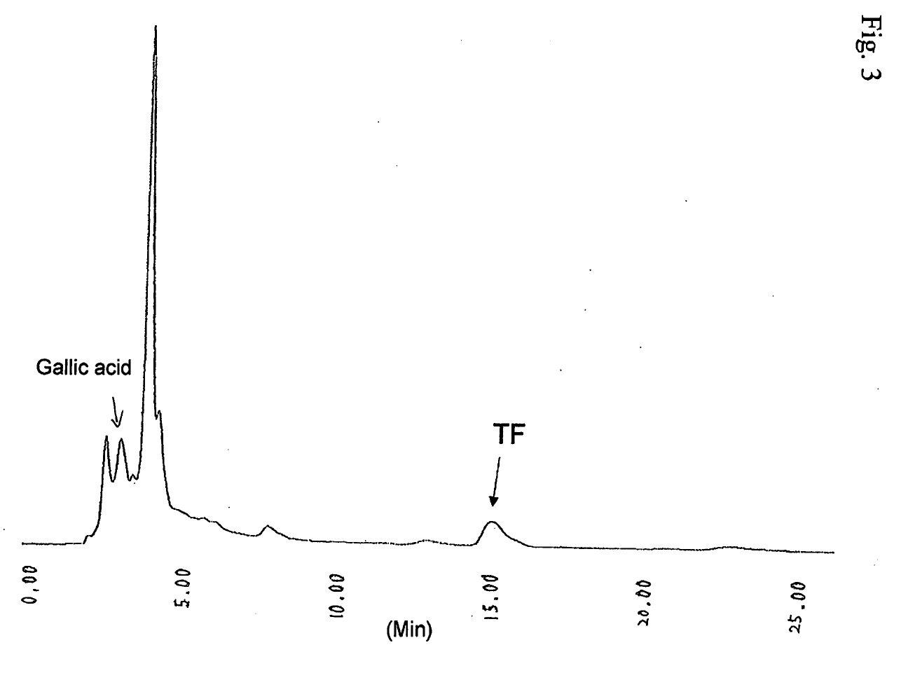

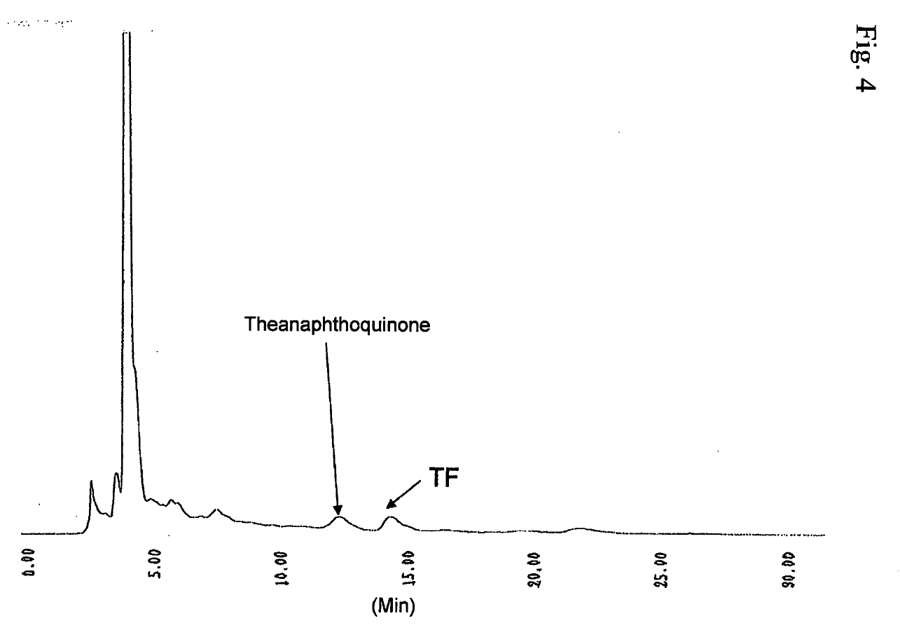

FIG. 3 shows the results of HPLC analysis of the reaction mixture at 9 minutes after start of the reaction. FIG. 4 shows the results of HPLC analysis of the reaction mixture upon completion of the reaction (28 min).

A peak for theaflavin was observed in HPLC analysis 9 minutes after start of the reaction. This demonstrated that synthesis reaction is possible even when using an ethyl acetate extract instead of a water extract, so that the reaction volume can be reduced by concentrating the materials. In addition, a peak for theanaphthoquinone produced by oxidation of theaflavin was observed in HPLC analysis upon completion of the reaction (28 min). This demonstrated that the produced theaflavin is further oxidized and converted to theanaphthoquinone.

### Example 3

For this experiment, the amount of reaction starting material was increased 10-fold compared with Example 2, and the theaflavin production was examined during a reaction time of 9 minutes.

A 241 mg of the bancha extract obtained in Example 2 (the 241 mg of bancha extract having an EC content of 43 mg, an ECG content of 11 mg, an EGC content of 49 mg and an EGCG content of 54 mg) was dissolved in 185 ml of water, and then 3 ml of 3% hydrogen peroxide and 24 ml of tea cell culture were added and reaction was carried out for 9 minutes. Extraction was performed 3 times with ethyl acetate after the reaction. Elution was performed with methanol in SephadexLH-20 column chromatography to obtain 17.2 mg of theaflavin.

This demonstrated that even when increasing the amount of reaction starting material the production of theaflavin was possible with a short reaction time (9 min).

### Example 4

Since the total amount of epicatechin and epicatechin-3-O-gallate was small with respect to the total amount of epigallocatechin and epigallocatechin-3-O-gallate in the bancha extract, epicatechin was replenished so that the (total epicatechin and epicatechin-3-O-gallate): (total epigallocatechin and epigallocatechin-3-O-gallate) ratio in the bancha extract for this experiment was equimolar, and the theaflavin production was examined.

A 700 ml of bancha extract (a filtrate obtained by 24 hours extraction of 5 g of dry bancha with 600 ml of water and filtration removal of the tea leaves, with the tea leaf washing procedure during filtration resulting in 700 ml of extract) was extracted 3 times with ethyl acetate. The ethyl acetate extract was concentrated, to obtain 1.82 g of bancha extract. A 203.7 mg portion of the bancha extract (the bancha extract having an EC content of 36.5 mg, an ECG content of 9.2 mg, an EGC content of 41.6 mg and an EGCG content of 45.5 mg) and 16.7 mg of epicatechin (Sigma) were dissolved in 185 ml of water, and then 3 ml of 3% hydrogen peroxide and 12 ml of tea cell culture were added and reaction was carried out for 12 minutes. Extraction was performed 3 times with ethyl acetate after the reaction. This was eluted with methanol in Sephadex LH-20 column chromatography to obtain 52.5 mg of theaflavin.

The yield calculated in the following manner from the amount of EGC (41.6 mg) in the bancha extract was 68.6%.

Yield based on EGC in bancha extract (%):

Produced TF 52.5 mg÷(EGC content in bancha extract 41.55 mg÷EGC molecular weight 306×TF molecular weight 564)×100=68.6%

By replenishing only epicatechin that was considered to be deficient in the bancha extract, it was possible to selectively obtain theaflavin at high yield with a short reaction time.

### Example 5

For this experiment, the amount of epicatechin was further replenished so that the (total epicatechin and epicatechin-3-O-gallate): (total epigallocatechin and epigallocatechin-3-O-gallate) ratio in the bancha extract was 1.3:1, and the theaflavin production was examined.

A 700 ml of bancha extract (a filtrate obtained by 24 hr extraction of 5 g of dry bancha with 600 ml of water and filtration removal of the tea leaves, with the tea leaf washing procedure during filtration resulting in 700 ml of extract) was extracted 3 times with ethyl acetate. The ethyl acetate extract was concentrated, to obtain 1.82 g of bancha extract. A 24.1 mg of the bancha extract (the 24.1 mg of bancha extract having an EC content of 4.3 mg, an ECG content of 1.1 mg, an EGC content of 4.9 mg and an EGCG content of 5.4 mg) and 4 mg of epicatechin were dissolved in 18.5 ml of water, and then 0.3 ml of 3% hydrogen peroxide and 1.2 ml of tea cell culture were added and reaction was carried out for 12 minutes. Extraction was performed 3 times with ethyl acetate after the reaction. This was eluted with methanol in Sephadex LH-20 column chromatography to obtain 13.8 mg of theaflavin.

The yield calculated in the following manner from the amount of EGC (4.9 mg) in the bancha extract was 152.2%. This means that theaflavin had been produced in excess of the amount of theaflavin producible from the EGC originally present in the bancha extract, suggesting that the deficient EGC with respect to EC was supplied from EGCG.

Yield based on EGC amount (4.9 mg) in bancha extract (%):

Produced TF 13.75 mg÷(EGC content in bancha extract 4.9 mg÷EGC molecular weight 306×TF molecular weight 564)×100)=152.2%

Assuming that EGCG in the bancha extract was converted to EGC with 100% conversion efficiency and participated in TF production, the yield (%) was calculated to be 87.5% as shown below. This suggested that it is possible to utilize all of the EC, ECG, EGC and EGCG in bancha extract as material for theaflavin by replenishing in excess only the epicatechin which may be deficient in the bancha extract.

Yield based on amounts of EGC (4.9 mg) and EGCG (5.43 mg) in bancha extract:

EGC content in bancha extract 4.9 mg÷EGC molecular weight 306=0.016 mmol

EGCG content in bancha extract 5.43 mg÷EGCG molecular weight 458=0.01186 mmol

Produced TF 13.75 mg÷((EGC content in bancha extract 0.016 mmol+EGCG content in bancha extract 0.01186 mmol)×TF molecular weight 564)×100)=87.5% yield

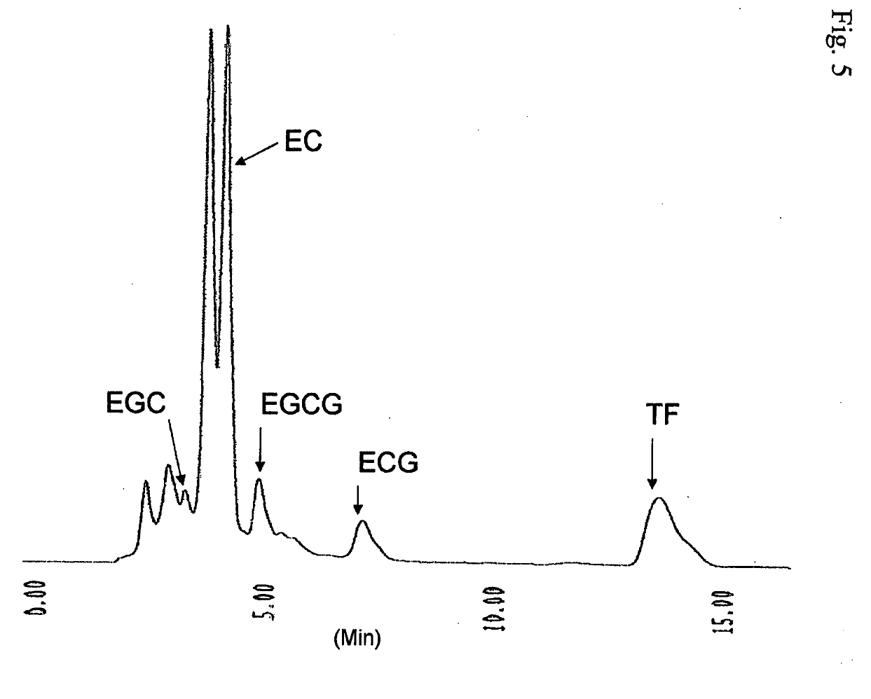

FIG. 5 shows the results of HPLC analysis of the reaction mixture at 5 minutes after start of the reaction. A peak for theaflavin (TF) is observed together with the peaks for EC, ECG, EGC and EGCG. This suggested that theaflavin (TF) synthesis reaction is a very rapid reaction.

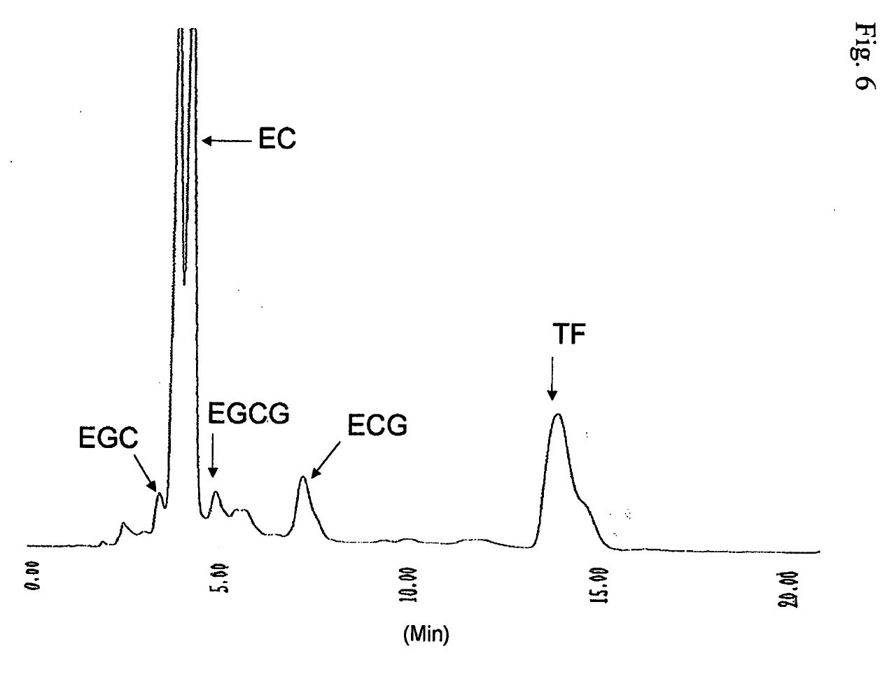

FIG. 6 shows the results of HPLC analysis of an ethyl acetate extract of the reaction mixture upon completion of the reaction (12 min). The EGC and EGCG were consumed by synthesis of theaflavin (TF).

### INDUSTRIAL APPLICABILITY

According to the method of the invention it is possible to produce theaflavin, which has many excellent physiologically active properties, in a highly selective manner at high yield, and to therefore obtain it efficiently without relying on extraction from black tea. In addition, since an inexpensive material such as bancha is used to produce theaflavin by a rapid and simple method, it is possible to produce theaflavin less expensively. The method of the invention can therefore contribute to future developments in theaflavin research, and can also potentially contribute to industry since it makes possible industrial mass production of theaflavins.

1. A method for selective production of theaflavin whereby a processed plant extract containing epicatechin, epigallocatechin, epicatechin-3-O-gallate and epigallocatechin-3-O-gallate is combined with a plant cell culture having peroxidase activity for selective production of theaflavin.

2. The method for producing theaflavin according to claim 1, wherein the plant is tea leaves and/or stems.

3. The method for producing theaflavin according to claim 1, wherein the processed plant is unfermented tea.

4. The method for producing theaflavin according to claim 1, wherein the plant cell culture is a culture of tea cells.

5. The method for producing theaflavin according to claim 1, characterized by further adding hydrogen peroxide.

6. The method for producing theaflavin according to claim 1, characterized by further adding epicatechin.

7. Cultured plant cells for the method for producing theaflavin according to claim 1.

8. The method for producing theaflavin according to claim 2, wherein the plant cell culture is a culture of tea cells.

9. The method for producing theaflavin according to claim 3, wherein the plant cell culture is a culture of tea cells.

10. The method for producing theaflavin according to claim 2, characterized by further adding hydrogen peroxide.

11. The method for producing theaflavin according to claim 3, characterized by further adding hydrogen peroxide.

12. The method for producing theaflavin according to claim 4, characterized by further adding hydrogen peroxide.

13. The method for producing theaflavin according to claim 8, characterized by further adding hydrogen peroxide.

14. The method for producing theaflavin according to claim 9, characterized by further adding hydrogen peroxide.

15. The method for producing theaflavin according to claim 2, characterized by further adding epicatechin.

16. The method for producing theaflavin according to claim 3, characterized by further adding epicatechin.

17. The method for producing theaflavin according to claim 4, characterized by further adding epicatechin.

18. The method for producing theaflavin according to claim 5, characterized by further adding epicatechin.

19. The method for producing theaflavin according to claim 8, characterized by further adding epicatechin.

20. The method for producing theaflavin according to claim 9, characterized by further adding epicatechin.

21. The method for producing theaflavin according to claim 10, characterized by further adding epicatechin.

22. The method for producing theaflavin according to claim 11, characterized by further adding epicatechin.

23. The method for producing theaflavin according to claim 12, characterized by further adding epicatechin.

24. The method for producing theaflavin according to claim 13, characterized by further adding epicatechin.

25. The method for producing theaflavin according to claim 14, characterized by further adding epicatechin.
